(19) United States
(12) Patent Application Publication
(10) Pub. No.: US 2024/0079096 A1
(43) Pub. Date: Mar. 7, 2024

(54) MIXING MODEL TO DETERMINE THE COMPOSITION OF PRODUCED WATER USING OXYGEN ISOTOPE RATIO AND CHLORIDE CONCENTRATION

(71) Applicant: Saudi Arabian Oil Company, Dhahran (SA)

(72) Inventor: Sattam S. Mutairi, Al Khobar (SA)

(21) Appl. No.: 17/901,473

(22) Filed: Sep. 1, 2022

(51) Int. Cl. G16C 20/20

(52) U.S. Cl. CPC G16C 20/20

(57) ABSTRACT

The disclosure relates to methods for determining an amount of seawater, an amount of aquifer water, and an amount of original reservoir water in a produced water sample using oxygen isotope ratios and chloride concentrations. An ¹⁸O/¹⁶O ratio (δ¹⁸O) and a chloride concentration are measured for a seawater sample, an aquifer water sample, and an original reservoir water sample and are used to make a mixing model. δ¹⁸O and a chloride concentration are measured in a produced water sample and the mixing model can be used to determine the amounts of each constituent in the produced water sample.

### FIELD

The disclosure relates to methods for determining an amount of seawater, an amount of aquifer water, and an amount of original reservoir water in a produced water sample using oxygen isotope ratios and chloride concentrations. An ¹⁸O/¹⁶O ratio (δ¹⁸O) and a chloride concentration are measured for a seawater sample, an aquifer water sample, and an original reservoir water sample and are used to make a mixing model. δ¹⁸O and a chloride concentration are measured in a produced water sample and the mixing model can be used to determine the amounts of each constituent in the produced water sample.

### BACKGROUND

Seawater and/or aquifer water can be used for drilling and enhanced oil recovery operations. Reservoirs can contain seawater from power injectors, aquifer water from gravity injections and residual reservoir connate water. Distinguishing between produced waters and in-situ water formation can be difficult due to the injection of aquifer waters and/or seawater. Often, chemical analyses alone (e.g. measurement of Cl concentration) has not been successful in distinguishing water sources.

### SUMMARY

The disclosure relates to methods for determining an amount of seawater, an amount of aquifer water, and an amount of original reservoir water in a produced water sample using oxygen isotope ratios and chloride concentrations. An ¹⁸O/¹⁶O ratio (δ¹⁸O) and a chloride concentration are measured for a seawater sample, an aquifer water sample, and an original reservoir water sample and are used to make a mixing model. δ¹⁸O and a chloride concentration are measured in a produced water sample and the mixing model can be used to determine the amounts of each constituent (seawater, aquifer water and/or original reservoir water) in the produced water sample.

The methods of the disclosure can enable the determination of the source of produced water and the effect of seawater and aquifer water on original in-situ waters. The methods of the disclosure can be used to make operational decisions enabling time and cost savings during drilling and production operations. The methods can be used to identify breakthrough of injection water, monitor water flooding and predict potential scaling problems caused by mixing of incompatible waters.

The methods of the disclosure can be used to analyze a mixture of waters with different salinity (e.g. low salinity aquifer water, moderate salinity seawater and/or high salinity reservoir water) which, in general, cannot be distinguished by chemical analysis. The methods of the disclosure can be compatible with chemical reactions caused by mixing of incompatible waters and chemicals such as scale inhibitors, which may alter the composition of produced water.

The methods of the disclosure employ major cations and anions and the isotope composition of oxygen (¹⁸O/¹⁶O) in original reservoir water, aquifer water and injected seawater as each water has distinct geochemistry. Oxygen isotopes can be measured with relatively high accuracy and are generally stable over production timescales. Chloride accounts for over 90% of the total anion charge in many water samples and can be accurately measured and the concentration is not readily altered. The methods of the disclosure do not rely on hydrogen isotope ratios (deuterium/hydrogen “D/H”), thereby avoiding any impact on D/H ratios caused by water-rock interactions in deep reservoir water.

In a first aspect, the disclosure provides a method, including measuring an ¹⁸O/¹⁶O ratio (δ¹⁸O) and a chloride concentration for each of a seawater sample, an aquifer water sample, and an original reservoir water sample; measuring δ¹⁸O and a chloride concentration in a produced water sample; using the measured δ¹⁸O and chloride concentrations for the aquifer water sample, the seawater sample and the original reservoir water sample to make a model; and using the model to determine an amount of seawater, an amount of aquifer water, and an amount of original reservoir water in the produced water sample. The produced water sample includes at least one member selected from the group consisting of the seawater, the aquifer water, and the original reservoir water.

In some embodiments, the model is for a well or formation (t) and is made using the equations

$${V}_{A} * Cl ( \frac{mg}{L} ) {x}_{Sewater} ( t ) + \text{} {V}_{B} * Cl ( \frac{mg}{L} ) {y}_{Aquifer water} ( t ) + {V}_{C} * Cl ( \frac{mg}{L} ) {z}_{Reservoir water} ( t ) = {V}_{D} * Cl ( \frac{mg}{L} ) ;$$

$$and$$

$$\frac{\begin{matrix}{V}_{A} * {W}_{H 2 O - Cl} {δ}^{18} O {x}_{Sewater} ( t ) + {V}_{B} * {W}_{H 2 O - Cl} {δ}^{18} O {y}_{Aquifer water} ( t ) + \\ {V}_{C} * {W}_{H 2 O - Cl} {δ}^{18} O {z}_{Reservoir water} ( t )\end{matrix}}{\begin{matrix}{V}_{A} * {W}_{H 2 O - Cl} {x}_{Sewater} ( t ) + {V}_{B} * {W}_{H 2 O - Cl} {y}_{Aquifer water} ( t ) + \\ {V}_{C} * {W}_{H 2 O - Cl} {z}_{Reservoir water} ( t ) = {V}_{D} * {δ}^{18} O ;\end{matrix}}$$

$$wherein :$$

$$C1 ( \frac{mg}{L} ) {x}_{S e w a t e r} , C1 ( \frac{mg}{L} ) {y}_{Aquifer water} and Cl ( \frac{mg}{L} ) {z}_{Reservoir water}$$

are the amounts of chloride in the seawater, aquifer water and original reservoir water respectively;

V_{A}, V_{B} and V_{C} are the relative volume fractions of the seawater, aquifer water and original reservoir water respectively;

V_{D} is the mixing proportion used in the model;

W_{H2O—Cl} is the mass of water calculated by subtracting chloride per unit volume of liquid in grams per liter; and

δ¹⁸O x_{sewater}, δ¹⁸O y_{Aquifer water}, and δ¹⁸O z_{Reservoir water} are the oxygen isotope ratios in the seawater, aquifer water and original reservoir water, respectively.

In some embodiments, W_{H2O—Cl} is calculated as

$${W}_{H 2 O - Cl} ( \frac{g}{L} ) = 1 , 000 - Slope ( {H}_{2} O vs Cl ) produced water * [ Cl , \frac{mg}{L} ] .$$

wherein Slope (H₂O vs Cl) produced water is the slope from a linear trend line fit to H₂O (mg/L) versus Cl (mg/L) for produced water samples.

In some embodiments, an equation to calculate W_{H2O—Cl} is obtained by fitting a linear trend line to chloride concentration values versus

$${W}_{H2O} ( \frac{g}{L} ) = \frac{999 * specific gravity - TDS}{1000}$$

TDS is the total dissolved solids calculated by the sum of all dissolved ions; and

the chloride concentration values, specific gravity and TDS are obtained from the produced water samples.

In some embodiments: the model includes first, second and third endpoints; V_{A}, V_{B} and V_{C} correspond to relative volume percentages of the seawater sample, the aquifer water sample, and the original reservoir water sample, respectively; the first endpoint corresponds to V_{A}=1, V_{B}=0 and V_{C}=0; the second endpoint corresponds to V_{A}=0; V_{B}=1 and V_{C}=0; and the third endpoint corresponds to V_{A}=0, V_{B}=0 and V_{C}=1.

In some embodiments: the model includes first, second and third outer lines that connect the endpoints to define a triangle; the first outer line corresponds to V_{A} being equal to zero while varying V_{B} and V_{C}; the second outer line corresponds to V_{B} being equal to zero while varying V_{A} and V_{C}; and the third outer line corresponds to V_{C} being equal to zero while varying V_{A} and V_{B}.

In some embodiments: the model includes grid lines that connect the outer lines; and each grid lines corresponds to one member selected from the group consisting of V_{A}, V_{B} and V_{C} being at a constant value between 0 and 1 and varying the other two members selected from the group consisting of V_{A}, V_{B} and V_{C}.

In some embodiments: each endpoint corresponds to a mixture including only one member selected from the group consisting of the seawater sample, the aquifer water sample, and the original reservoir water sample; each outer line corresponds to a mixture including two members selected from the group consisting of the seawater sample, the aquifer water sample, and the original reservoir water sample; and each grid line corresponds to a mixture including the seawater sample, the aquifer water sample, and the original reservoir water sample.

In some embodiments: a produced water sample including only one member selected from the group consisting of the seawater sample, the aquifer water sample, and the original reservoir water sample plots close to its corresponding endpoint; a produced water sample including only two of members selected from the group consisting of the seawater sample, the aquifer water sample, and the original reservoir water sample plots along the outer lines at a position corresponding to an amount of each of the two members; and a produced water sample including the seawater sample, the aquifer water sample, and the original reservoir water sample plots in at a position within the triangle corresponding to an amount of each of the seawater sample, the aquifer water sample, and the original reservoir water sample.

In some embodiments: the model includes endpoints that correspond to mixtures including only one member selected from the group consisting of the seawater sample, the aquifer water sample, and the original reservoir water sample; the model includes outer lines connecting the endpoints that correspond to mixtures including only two members selected from the group consisting of the seawater sample, the aquifer water sample, and the original reservoir water sample; the outer lines define a triangle; and the model includes grid lines that connect the outer lines that correspond to mixtures including the seawater sample, the aquifer water sample, and the original reservoir water sample.

In some embodiments: a produced water sample includes only one member selected from the group consisting of the seawater sample, the aquifer water sample, and the original reservoir water sample plots close to the corresponding endpoint; a produced water sample including two members selected from the group consisting of the seawater sample, the aquifer water sample, and the original reservoir water sample plots along the outer lines at a position corresponding to an amount of each of the two members; and a produced water sample including the seawater sample, the aquifer water sample, and the original reservoir water sample plots at a position within the triangle corresponding to an amount of each of the seawater sample, the aquifer water sample, and the original reservoir water sample.

In some embodiments, the seawater has a concentration of chloride of 5000 mg/L to 40000 mg/L.

In some embodiments, the seawater has a δ¹⁸O of −5 to 7.

In some embodiments, the aquifer water has a concentration of chloride of 100 mg/L to 6000 mg/L.

In some embodiments, the aquifer water has a δ¹⁸O of −10 to 3.

In some embodiments, the original reservoir water has a concentration of chloride of 40000 mg/L to 250000 mg/L

In some embodiments, the original reservoir water has a δ¹⁸O of −2 to 8.

In some embodiments, the produced water is from a production well in fluid communication with an underground reservoir.

In some embodiments, the produced water is from a flowback operation. In some embodiments, the seawater is injected into the underground reservoir

### BRIEF DESCRIPTION OF THE FIGURES

FIG. 1 is a plot of chloride concentration versus water mass per volume.

FIG. 2 is a plot of chlorine concentration versus δ¹⁸O according to a mixing model.

FIG. 3 is a plot of chlorine concentration versus δ¹⁸O according to a mixing model.

FIG. 4 is a plot of chlorine concentration versus δ¹⁸O according to a mixing model with produced water samples plotted on the mixing model.

FIG. 5 is a plot of chlorine concentration versus δ¹⁸O according to a mixing model with produced water samples plotted on the mixing model.

### DETAILED DESCRIPTION

Oxygen isotope ratios can be reported relative to Vienna Standard Mean Ocean Water (VSMOW) using delta notation:

$${δ}^{18} O ( o / oo ) = 1000 [ \frac{{( \frac{{18}_{O}}{{16}_{O}} )}_{S A M P L E}}{{( \frac{{18}_{O}}{{16}_{O}} )}_{VSMOW}} - 1 ] \qquad ( 1 )$$

where ¹⁸O/¹⁶O are atomic abundance ratios.

Mixing proportions of chloride and oxygen isotopes for a well or formation (t) with three mixing components and relative volume percentages V_{A}, V_{B} and V_{C} yield the equations:

$${V}_{A} * Cl ( \frac{mg}{L} ) {x}_{Sewater} ( t ) + \text{} {V}_{B} * Cl ( \frac{mg}{L} ) {y}_{Aquifer water} ( t ) + {V}_{C} * Cl ( \frac{mg}{L} ) {z}_{Reservoir water} ( t ) = {V}_{D} * Cl ( \frac{mg}{L} ) \qquad ( 2 )$$

$$and$$

$$\frac{\begin{matrix}{V}_{A} * {W}_{H 2 O - Cl} {δ}^{18} O {x}_{Sewater} ( t ) + {V}_{B} * {W}_{H 2 O - Cl} {δ}^{18} O {y}_{Aquifer water} ( t ) + \\ {V}_{C} * {W}_{H 2 O - Cl} {δ}^{18} O {z}_{Reservoir water} ( t )\end{matrix}}{\begin{matrix}{V}_{A} * {W}_{H 2 O - Cl} {x}_{Sewater} ( t ) + {V}_{B} * {W}_{H 2 O - Cl} {y}_{Aquifer water} ( t ) + \\ {V}_{C} * {W}_{H 2 O - Cl} {z}_{Reservoir water} ( t ) = {V}_{D} * {δ}^{18} O\end{matrix}} \qquad ( 3 )$$

wherein: Cl (mg/L) X_{Sewater}, Cl (mg/L) y_{Aquifer water}, and Cl (mg/L) z_{Reservoir water} are the amounts of chloride in the seawater, aquifer water and original reservoir water respectively; V_{A}, V_{B} and V_{C} are the relative volume fractions of the seawater, aquifer water and original reservoir water, respectively; V_{D} is the mixing proportion used in the model; W_{H2O—Cl} is the mass of water calculated by subtracting chloride per unit volume of liquid in grams per liter; and δ¹⁸O x_{Sewater}, δ¹⁸O y_{Aquifer water}, and δ¹⁸O z_{Reservoir water} are the oxygen isotope ratios in the seawater, aquifer water and original reservoir water, respectively. W_{H2O} is unique for each water source (seawater, aquifer water and original reservoir water). Typically, the value of V_{D} is 1.

Conversion of the mass of ions (e.g., chloride) from milligrams per liter of fluid to milligrams per kg of water for use in equation (2) can be performed using the equation:

M(mg/kg water)=M(mg/L)/W_{H2O—Cl} (4)

where M (mg/kg water) and M (mg/L) refer to the mass of chloride in mg/kg water and mg/L, respectively. W is the total amount of water with all dissolved ions. The mass of chloride is divided by the total water (W) to specifically get mg chloride per L of water.

The equations assume ideal mixing but incorporate small corrections for the different concentrations of water in each of the endpoints (seawater, aquifer water and original reservoir water). Endpoint water concentrations can be estimated using the equation:

$${W}_{H 2 O - Cl} ( \frac{g}{L} ) = 1 , 000 - Slope ( {H}_{2} O vs Cl ) produced water * [ Cl , \frac{mg}{L} ] \qquad ( 5 )$$

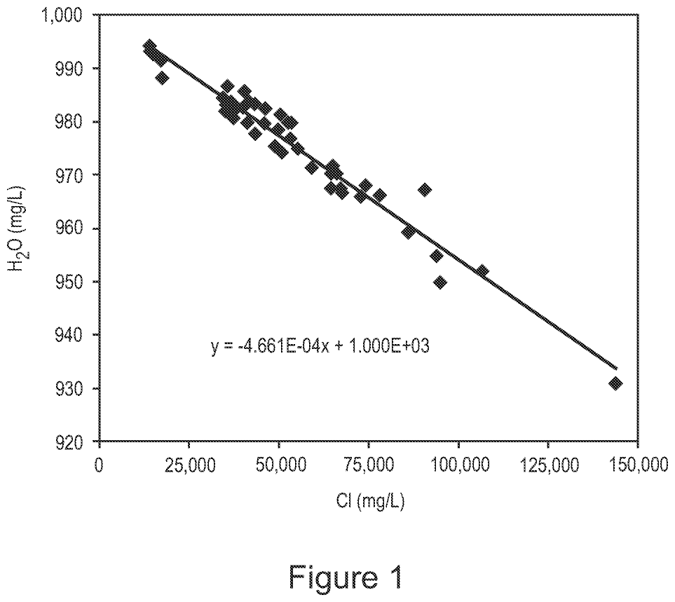

which is obtained by fitting a linear trend line to chloride concentration values versus

$${W}_{H2O} ( \frac{g}{L} ) = \frac{999 * specific gravity}{1000} \qquad ( 6 )$$

where TDS is the total dissolved solids calculated by the sum of all dissolved ions. Equation (5) is recalculated for each field or well. FIG. 1 shows a plot of chloride concentration versus W_{H2O} from equation (6) with a linear trend line to obtain equation (5) as shown in FIG. 1. The parameters in equation (5) are obtained from seawater, aquifer and original reservoir waters. Equation (6) is calculated using produced water samples. In this case, Slope (H₂O vs Cl) produced water has a value of 4.4661×10⁻⁴.

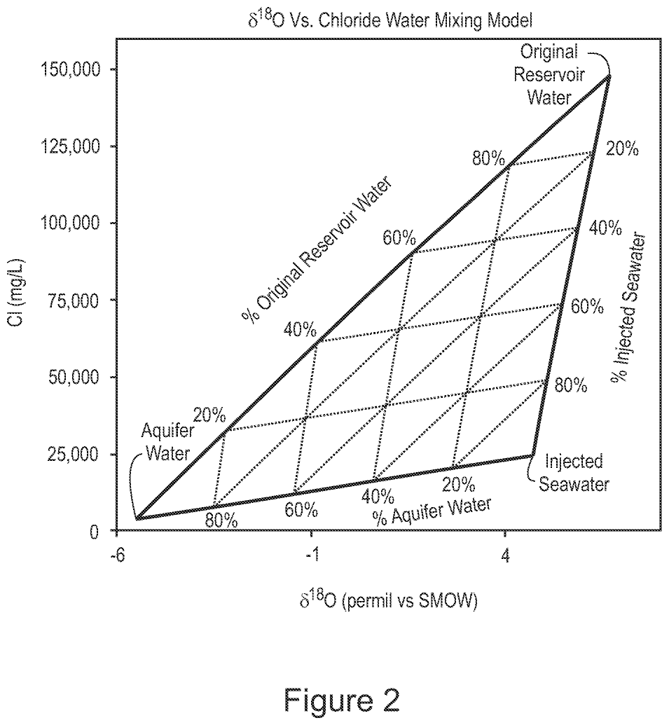

The chloride concentrations and oxygen isotope ratios for seawater, aquifer water and original reservoir water should be obtained for a given reservoir. Once obtained, the values can be used to construct the model. Such a model is depicted in FIGS. 2-5. These figures are explained in more detail below. In FIGS. 2-5, the endpoints correspond to the vertices of the triangle.

Generally, the model contains three endpoints, where each endpoint corresponds to one of the three components (the seawater, the aquifer water and the original reservoir water). Each endpoint can be determined by setting one of the volume fractions V_{A}, V_{B} and V_{C}, corresponding to the fraction of seawater, aquifer water and original reservoir water respectively, to 1, while setting to other two volume fractions to 0. Specifically, V_{A}=1, V_{B}=0 and V_{C}=0; V_{A}=0; V_{B}=1 and V_{C}=0; and V_{A}=0, V_{B}=0 and V_{C}=1 in equations 2 and 3 provide the three endpoints.

The model additionally contains outer lines connecting the endpoints that correspond to mixtures of two of the components. The outer lines can be obtained by setting one of V_{A}, V_{B} and V_{C} to 0 and varying the other two parameters. The model can further contain grid lines that connect the outer lines that correspond to mixtures of the three components. The grid lines can be obtained by holding one of V_{A}, V_{B} and V_{C} at a constant value between 0 and 1 and not equal to 0 or 1 and varying the other two components.

Generally, a produced water sample containing only one of the components will plot close to the corresponding endpoint. A produced water sample containing only two of the components will plot along the outer lines at a position corresponding to an amount of each of the two components. A produced water sample containing all three of the components will plot in the center of the triangle at a position corresponding to an amount of each of the three components.

In general, chloride concentrations can be measured using any appropriate technique. As an example, in some embodiments, chloride concentrations are measured by potentiometric titration, such as, for example, potentiometric titration performed with silver nitrate.

Generally, cations and/or anions can be measures using any appropriate technique. As an example, in some embodiments, cations and/or anions are measured by inductively coupled plasma atomic emission spectroscopy (ICP-AES).

Typically, isotope concentrations in a water sample can be measured using any appropriate technique. As an example, in certain embodiments, the amount of ¹⁶O and/or ¹⁸O is measured using a wavelength-scanned cavity ring-down spectroscopy (WS-CRDS). As another example, in certain embodiments, the amount of ¹⁶O and/or ¹⁸O can be measured using mass spectrometry.

In certain embodiments, the concentration of chloride in the seawater, the aquifer water and/or the original reservoir water depends on the geographical location of the water. In some embodiments, the seawater has a concentration of chloride of at least 5000 (e.g., at least 10000, at least 15000, at least 20000, at least 25000, at least 30000, at least 35000) mg/L and at most 40000 (e.g., at most 35000, at most 30000, at most 25000, at most 20000, at most 15000, at most 10000) mg/L. In some embodiments, the aquifer water has a concentration of chloride of at least 100 (e.g., at least 500, at least 1000, at least 1500, at least 2000, at least 2500, at least 3000, at least 3500, at least 4000, at least 4500, at least 5000, at least 5500) mg/L and at most 6000 (e.g., at most 5500, at most 5000, at most 4500, at most 4000, at most 3500, at most 3000, at most 2500, at most 2000, at most 1500, at most 1000, at most 500) mg/L. In some embodiments, the original reservoir water has a concentration of chloride of at least 40000 (e.g., at least 50000, at least 100000, at least 150000, at least 200000) mg/L and at most 250000 mg/L (e.g., at most 200000, at most 150000, at least 100000, at least 50000) mg/L.

In certain embodiments, the δ¹⁸O of the seawater, the aquifer water and/or the original reservoir water depends on the geographical location of the water. In certain embodiments, the seawater has a δ¹⁸O of at least −5 (e.g., at least −4, at least −3, at least −2, at least −1, at least 0, at least 1, at least 2, at least 3, at least 4, at least 5) and at most 7 (e.g., at most 6, at most 5, at most 4, at most 3, at most 2, at most 1, at most 0, at most −1, at most −2, at most −3). In certain embodiments, the aquifer water has a δ¹⁸O of at least −10 (e.g., at least −9, at least −8, at least −7, at least −6, at least −5) and at most −3 (e.g., at most −4, at most −5, at most −6, at most −7, at most −8). In certain embodiments, the original reservoir water has a δ¹⁸O of at least −2 (e.g., at least −1, at least 0, at least 1, at least 2, at least 3, at least 4, at least 5, at least 6) and at most 8 (e.g., at most 7, at most 6, at most 5, at most 4, at most 3, at most 2, at most 1, at most 0).

### EXAMPLES

Example 1—Construction of Mixing Model

Chloride and cation concentrations and δ¹⁸O were measured for samples of seawater, aquifer water and original reservoir water. Cation concentrations for produced water samples were measured by ICP-AES (7850 ICP-MS, Agilent Technologies and ICP-OES spectrometer 240FS AA, Agilent Technologies) for use in equation (6).

Chloride concentrations were determined by potentiometric titration with silver nitrate solution with a glass silver/silver chloride electrode. During titration an electronic voltmeter (Mettler DL-77 Auto Titrator) was used to detect the change in potential between the two electrodes. The end point of the titration was detected by the instrument. Quality control check standard were run with the samples to ensure accuracy of results. The measurements were generally accurate to ±3% and were rejected if they differed by more than ±5% from the expected values.

The chloride concentrations for samples of seawater, aquifer water and original reservoir water were used to calculate W_{H2O—Cl} using equation (5).

δ¹⁸O were measured using both wavelength-scanned cavity ring-down spectroscopy mass spectrometry.

δ¹⁸O of the samples were measured using a Picarro L2130-i Laser Cavity Ring-Down Spectroscopy (CRDS) instrument. 5 μl of sample was injected into a vaporizer a total of 8 times. The temperature and pressure were kept constant at 110° C. and 3.5 psig inside the line to ensure total and instantaneous vaporization of the water without isotopic fractionation.

For mass spectrometry measurements, 5 ml aliquots of sample were placed in 25 ml glass vials, attached to a vacuum manifold and immersed in a water bath regulated to 18.0±0.2° C. The manifold was used to remove air and add 0.7-0.9 bars of carbon dioxide. Isotopic equilibration between carbon dioxide and water was achieved overnight. The equilibrated gases were passed over acetone slush (−90-−96° C.) to remove water vapor. The gases were then introduced into a dual-inlet Finnigan-MAT Delta S stable isotope ratio mass spectrometer using automated valves. Water standards (USGS-48, USGS-47, USGS-48, USGS-50, PRIMARY VSMOW STANDARD, and three other lab made standards) were used to calibrate the isotope ratios and report them relative to standard mean ocean water (SMOW) using delta notation.

Reproducibilities, estimated by pooling standard deviations for replicate analyses of standards were ±0.13% for δ¹⁸O for both instruments.

The oxygen isotope ratios, chloride concentrations and calculated total water for the seawater, aquifer and original reservoir water samples measured are presented in table 1.

TABLE 1
Oxygen isotope ratios and chloride concentrations
for source water samples

| | | | H₂O g/L (calculated |
|---|---|---|---|
| Component | δ¹⁸O | Cl (mg/L) | from equation (5)) |
| Seawater | 4.7 | 24,500 | 988.58 |
| Aquifer water | −5.5 | 3,400 | 998.42 |
| Original reservoir | 6.7 | 148,000 | 931.02 |
| water | | | |

The values of V_{A}, V_{B} and V_{C} were each set to 1 while the other two were set to 0 in equations 2 and 3 to provide the values for the endpoints of the model, as shown in table 2.

TABLE 2
Oxygen isotope ratios and chloride concentrations model endpoints

| | Fraction of | Fraction of | | |
|---|---|---|---|---|
| Fraction of | aquifer | original reservoir | | Cl |
| seawater (V_{A}) | water (V_{B}) | water (V_{C}) | δ¹⁸O | (mg/L) |
| 1 | 0 | 0 | 4.7 | 24,500 |
| 0 | 1 | 0 | −5.5 | 3,400 |
| 0 | 0 | 1 | 6.7 | 148,000 |

The outer lines and grid lines of the model were calculated by varying the values of V_{A}, V_{B} and V_{C} in equations 2 and 3. Tables 3 and 4 shows the values of δ¹⁸O and Chloride concentration used to make the outer lines and the grid lines, respectively.

TABLE 3
Oxygen isotope ratios and chloride
concentrations model outer lines

| | Fraction of | Fraction of | | |
|---|---|---|---|---|
| Fraction of | aquifer | original reservoir | | Cl |
| seawater (V_{A}) | water (V_{B}) | water (V_{C}) | δ¹⁸O | (mg/L) |
| 1 | 0 | 0 | 4.70 | 24500.0 |
| 0.9 | 0.1 | 0 | 3.67 | 22390.0 |
| 0.8 | 0.2 | 0 | 2.64 | 20280.0 |
| 0.7 | 0.3 | 0 | 1.62 | 18170.0 |
| 0.6 | 0.4 | 0 | 0.60 | 16060.0 |
| 0.5 | 0.5 | 0 | −0.43 | 13950.0 |
| 0.4 | 0.6 | 0 | −1.44 | 11840.0 |
| 0.3 | 0.7 | 0 | −2.46 | 9730.0 |
| 0.2 | 0.8 | 0 | −3.48 | 7620.0 |
| 0.1 | 0.9 | 0 | −4.49 | 5510.0 |
| 0 | 1 | 0 | −5.50 | 3400.0 |
| 0 | 0.9 | 0.1 | −4.35 | 17860.0 |
| 0 | 0.8 | 0.2 | −3.19 | 32320.0 |
| 0 | 0.7 | 0.3 | −2.02 | 46780.0 |
| 0 | 0.6 | 0.4 | −0.82 | 61240.0 |
| 0 | 0.5 | 0.5 | 0.39 | 75700.0 |
| 0 | 0.4 | 0.6 | 1.61 | 90160.0 |
| 0 | 0.3 | 0.7 | 2.86 | 104620.0 |
| 0 | 0.2 | 0.8 | 4.12 | 119080.0 |
| 0 | 0.1 | 0.9 | 5.40 | 133540.0 |
| 0 | 0 | 1 | 6.70 | 148000.0 |
| 0.1 | 0 | 0.9 | 6.49 | 135650.0 |
| 0.2 | 0 | 0.8 | 6.28 | 123300.0 |
| 0.3 | 0 | 0.7 | 6.07 | 110950.0 |
| 0.4 | 0 | 0.6 | 5.87 | 98600.0 |
| 0.5 | 0 | 0.5 | 5.67 | 86250.0 |
| 0.6 | 0 | 0.4 | 5.47 | 73900.0 |
| 0.7 | 0 | 0.3 | 5.28 | 61550.0 |
| 0.8 | 0 | 0.2 | 5.08 | 49200.0 |
| 0.9 | 0 | 0.1 | 4.89 | 36850.0 |
| 1 | 0 | 0 | 4.70 | 24500.0 |

TABLE 4
Oxygen isotope ratios and chloride concentrations model grid lines

| | Fraction of | Fraction of | | |
|---|---|---|---|---|
| Fraction of | aquifer | original reservoir | | Cl |
| seawater (V_{A}) | water (V_{B}) | water (V_{C}) | δ¹⁸O | (mg/L) |
| 0.2 | 0.8 | 0 | −3.48 | 7620.0 |
| 0.2 | 0.7 | 0.1 | −2.31 | 22080.0 |
| 0.2 | 0.6 | 0.2 | −1.14 | 36540.0 |
| 0.2 | 0.5 | 0.3 | 0.06 | 51000.0 |
| 0.2 | 0.4 | 0.4 | 1.27 | 65460.0 |
| 0.2 | 0.3 | 0.5 | 2.49 | 79920.0 |
| 0.2 | 0.2 | 0.6 | 3.74 | 94380.0 |
| 0.2 | 0.1 | 0.7 | 5.00 | 108840.0 |
| 0.2 | 0 | 0.8 | 6.28 | 123300.0 |
| 0.4 | 0.6 | 0 | −1.44 | 11840.0 |
| 0.4 | 0.5 | 0.1 | −0.27 | 26300.0 |
| 0.4 | 0.4 | 0.2 | 0.93 | 40760.0 |
| 0.4 | 0.3 | 0.3 | 2.14 | 55220.0 |
| 0.4 | 0.2 | 0.4 | 3.36 | 69680.0 |
| 0.4 | 0.1 | 0.5 | 4.61 | 84140.0 |
| 0.4 | 0 | 0.6 | 5.87 | 98600.0 |
| 0.6 | 0.4 | 0 | 0.60 | 16060.0 |
| 0.6 | 0.3 | 0.1 | 1.79 | 30520.0 |
| 0.6 | 0.2 | 0.2 | 3.00 | 44980.0 |
| 0.6 | 0.1 | 0.3 | 4.23 | 59440.0 |
| 0.6 | 0 | 0.4 | 5.47 | 73900.0 |
| 0.8 | 0.2 | 0 | 2.64 | 20280.0 |
| 0.8 | 0.1 | 0.1 | 3.85 | 34740.0 |
| 0.8 | 0 | 0.2 | 5.08 | 49200.0 |
| 0 | 0.2 | 0.8 | 4.12 | 119080.0 |
| 0.1 | 0.2 | 0.7 | 3.93 | 106730.0 |
| 0.2 | 0.2 | 0.6 | 3.74 | 94380.0 |
| 0.3 | 0.2 | 0.5 | 3.55 | 82030.0 |
| 0.4 | 0.2 | 0.4 | 3.36 | 69680.0 |
| 0.5 | 0.2 | 0.3 | 3.18 | 57330.0 |
| 0.6 | 0.2 | 0.2 | 3.00 | 44980.0 |
| 0.7 | 0.2 | 0.1 | 2.82 | 32630.0 |
| 0.8 | 0.2 | 0 | 2.64 | 20280.0 |
| 0 | 0.4 | 0.6 | 1.61 | 90160.0 |
| 0.1 | 0.4 | 0.5 | 1.44 | 77810.0 |
| 0.2 | 0.4 | 0.4 | 1.27 | 65460.0 |
| 0.3 | 0.4 | 0.3 | 1.10 | 53110.0 |
| 0.4 | 0.4 | 0.2 | 0.93 | 40760.0 |
| 0.5 | 0.4 | 0.1 | 0.76 | 28410.0 |
| 0.6 | 0.4 | 0 | 0.60 | 16060.0 |
| 0 | 0.6 | 0.4 | −0.82 | 61240.0 |
| 0.1 | 0.6 | 0.3 | −0.98 | 48890.0 |
| 0.2 | 0.6 | 0.2 | −1.14 | 36540.0 |
| 0.3 | 0.6 | 0.1 | −1.29 | 24190.0 |
| 0.4 | 0.6 | 0 | −1.44 | 11840.0 |
| 0 | 0.8 | 0.2 | −3.19 | 32320.0 |
| 0.1 | 0.8 | 0.1 | −3.34 | 19970.0 |
| 0.2 | 0.8 | 0 | −3.48 | 7620.0 |
| 0 | 0.8 | 0.2 | −3.19 | 32320.0 |
| 0.1 | 0.7 | 0.2 | −2.17 | 34430.0 |
| 0.2 | 0.6 | 0.2 | −1.14 | 36540.0 |
| 0.3 | 0.5 | 0.2 | −0.11 | 38650.0 |
| 0.4 | 0.4 | 0.2 | 0.93 | 40760.0 |
| 0.5 | 0.3 | 0.2 | 1.96 | 42870.0 |
| 0.6 | 0.2 | 0.2 | 3.00 | 44980.0 |
| 0.7 | 0.1 | 0.2 | 4.04 | 47090.0 |
| 0.8 | 0 | 0.2 | 5.08 | 49200.0 |
| 0 | 0.6 | 0.4 | −0.82 | 61240.0 |
| 0.1 | 0.5 | 0.4 | 0.22 | 63350.0 |
| 0.2 | 0.4 | 0.4 | 1.27 | 65460.0 |
| 0.3 | 0.3 | 0.4 | 2.31 | 67570.0 |
| 0.4 | 0.2 | 0.4 | 3.36 | 69680.0 |
| 0.5 | 0.1 | 0.4 | 4.42 | 71790.0 |
| 0.6 | 0 | 0.4 | 5.47 | 73900.0 |
| 0 | 0.4 | 0.6 | 1.61 | 90160.0 |
| 0.1 | 0.3 | 0.6 | 2.67 | 92270.0 |
| 0.2 | 0.2 | 0.6 | 3.74 | 94380.0 |
| 0.3 | 0.1 | 0.6 | 4.80 | 96490.0 |
| 0.4 | 0 | 0.6 | 5.87 | 98600.0 |
| 0 | 0.2 | 0.8 | 4.12 | 119080.0 |
| 0.1 | 0.1 | 0.8 | 5.20 | 121190.0 |
| 0.2 | 0 | 0.8 | 6.28 | 123300.0 |

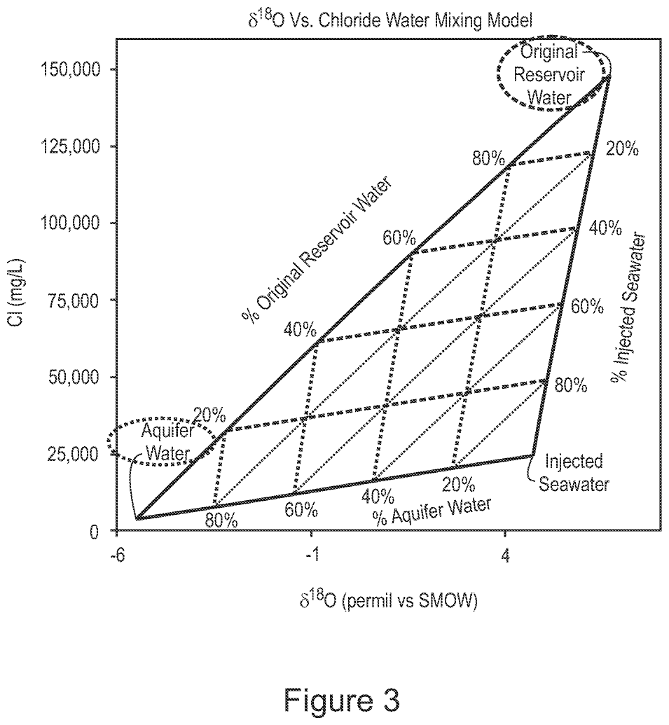

The mixing model is shown in FIG. 2. FIG. 3 shows the mixing model where the grid lines for the aquifer water and original reservoir water are dotted and dashed lines, respectively.

Example 2—Characterization of Produced Water Samples

The mixing model generated in Example 1 was used to determine the composition of produced water samples. Chloride concentrations and δ¹⁸O of 128 produced water samples from five different wells in the same field and reservoir taken during different field operations were determined using the methods described in Example 1.

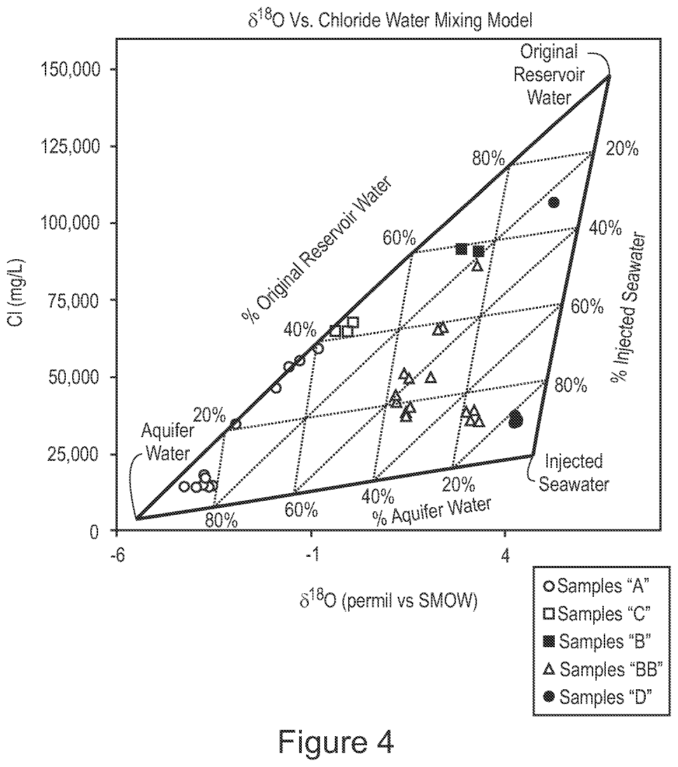

FIG. 4 shows the produced water samples plotted on the mixing model.

Produced water samples “A” plot very closed to the aquifer water end-member, suggesting that the produced water was composed almost entirely of injected aquifer water. However, a few samples plot along the line between the aquifer water and original reservoir water, which suggests mixing between the original reservoir and aquifer water sources.

Produced water samples “B” were nearly 60% original reservoir water, 20% injected seater and 20% aquifer water.

Produced water samples “BB” contained all three water sources and were divided into two clusters. Some of the “BB” samples contained mainly of injected seawater (over 70%). The other samples were distributed between the three sources. This indicates that seawater and aquifer water were injected into the original reservoir water, causing the produced water samples to possess all three water sources.

Produced water sample “C” plot along the aquifer water and original reservoir water line. Thus, this sample was composed of a mixture of aquifer water and original reservoir water with minimal effect of seawater.

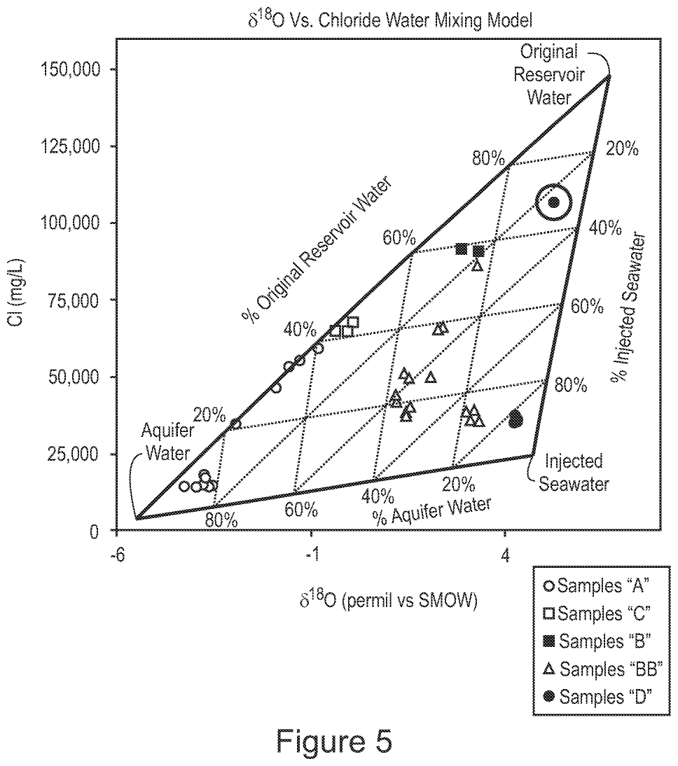

Produced water sample “D” plot along the mixing line between injected seawater and original reservoir water, suggesting that most of the samples was composed of injected seawater with small amounts of original reservoir water and fresh aquifer water. FIG. 5 shows FIG. 4 with one sample from “D” circled. This data point was identified using the model as about 63% original reservoir water, more than 30% injected seawater and less than 10% fresh aquifer water.

The mixing model was successfully applied to produced water to help predict and estimate the source of produced water and the effect of seawater and/or aquifer water on the original reservoir water. This information can help make quick field operation decisions, thereby saving time and reducing costs in drilling and production operations.

What is claimed:

1. A method, comprising:
measuring an ¹⁸O/¹⁶O ratio (δ¹⁸O) and a chloride concentration for each of a seawater sample, an aquifer water sample, and an original reservoir water sample;
measuring δ¹⁸O and a chloride concentration in a produced water sample;
using the measured δ¹⁸O and chloride concentrations for the aquifer water sample, the seawater sample and the original reservoir water sample to make a model; and
using the model to determine an amount of seawater, an amount of aquifer water, and an amount of original reservoir water in the produced water sample,
wherein the produced water sample comprises at least one member selected from the group consisting of the seawater, the aquifer water, and the original reservoir water.

2. The method of claim 1, wherein the model is for a well or formation (t) and is made using the equations
$${V}_{A} * Cl ( \frac{mg}{L} ) {x}_{Sewater} ( t ) + \text{} {V}_{B} * Cl ( \frac{mg}{L} ) {y}_{Aquifer water} ( t ) + {V}_{C} * Cl ( \frac{mg}{L} ) {z}_{Reservoir water} ( t ) = {V}_{D} * Cl ( \frac{mg}{L} ) ;$$
$$and$$
$$\frac{\begin{matrix}{V}_{A} * {W}_{H 2 O - Cl} {δ}^{18} O {x}_{Sewater} ( t ) + {V}_{B} * {W}_{H 2 O - Cl} {δ}^{18} O {y}_{Aquifer water} ( t ) + \\ {V}_{C} * {W}_{H 2 O - Cl} {δ}^{18} O {z}_{Reservoir water} ( t )\end{matrix}}{\begin{matrix}{V}_{A} * {W}_{H 2 O - Cl} {x}_{Sewater} ( t ) + {V}_{B} * {W}_{H 2 O - Cl} {y}_{Aquifer water} ( t ) + \\ {V}_{C} * {W}_{H 2 O - Cl} {z}_{Reservoir water} ( t ) = {V}_{D} * {δ}^{18} O ;\end{matrix}}$$
$$wherein :$$
$$C l ( \frac{mg}{L} ) {x}_{S e w a t e r} , C l ( \frac{mg}{L} ) {y}_{Aquifer water} and C l ( \frac{mg}{L} ) {z}_{Reservoir water}$$
are the amounts of chloride in the seawater, aquifer water and original reservoir water respectively;
V_{A}, V_{B} and V_{C} are the relative volume fractions of the seawater, aquifer water and original reservoir water respectively;
V_{D} is the mixing proportion used in the model;
W_{H2O—Cl} is the mass of water calculated by subtracting chloride per unit volume of liquid in grams per liter; and
δ¹⁸O x_{Sewater}, δ¹⁸O y_{Aquifer water}, and δ¹⁸O z_{Reservoir} water are the oxygen isotope ratios in the seawater, aquifer water and original reservoir water, respectively.

3. The method of claim 2, wherein W_{H2O—Cl} is calculated as
$${W}_{H 2 O - Cl} ( \frac{g}{L} ) = 1 , 000 - Slope ( {H}_{2} O vs Cl ) produced water * [ Cl , \frac{mg}{L} ] .$$
wherein Slope (H₂O vs Cl) produced water is the slope from a linear trend line fit to H₂O (mg/L) versus Cl (mg/L) for produced water samples.

4. The method of claim 3, wherein:
an equation to calculate W_{H2O—Cl} is obtained by fitting a linear trend line to chloride concentration values versus
$${W}_{H20} ( \frac{g}{L} ) = \frac{999 * specific gravity - TDS}{1000}$$
TDS is the total dissolved solids calculated by the sum of all dissolved ions; and
the chloride concentration values, specific gravity and TDS are obtained from the produced water samples.

5. The method of claim 2, wherein:
the model comprises first, second and third endpoints;
V_{A}, V_{B} and V_{C} correspond to relative volume percentages of the seawater sample, the aquifer water sample, and the original reservoir water sample, respectively
the first endpoint corresponds to V_{A}=1, V_{B}=0 and V_{C}=0;
the second endpoint corresponds to V_{A}=0; V_{B}=1 and V_{C}=0; and
the third endpoint corresponds to V_{A}=0, V_{B}=0 and V_{C}=1.

6. The method of claim 5, wherein:
the model comprises first, second and third outer lines that connect the endpoints to define a triangle;
the first outer line corresponds to V_{A} being equal to zero while varying V_{B} and V_{C};
the second outer line corresponds to V_{B} being equal to zero while varying V_{A} and V_{C}; and
the third outer line corresponds to V_{C} being equal to zero while varying V_{A} and V_{B}.

7. The method of claim 6, wherein:
the model comprises grid lines that connect the outer lines; and
each grid lines corresponds to one member selected from the group consisting of V_{A}, V_{B} and V_{C} being at a constant value between 0 and 1 and varying the other two members selected from the group consisting of V_{A}, V_{B} and V_{C}.

8. The method of claim 7, wherein:
each endpoint corresponds to a mixture comprising only one member selected from the group consisting of the seawater sample, the aquifer water sample, and the original reservoir water sample;
each outer line corresponds to a mixture comprising two members selected from the group consisting of the seawater sample, the aquifer water sample, and the original reservoir water sample; and
each grid line corresponds to a mixture comprising the seawater sample, the aquifer water sample, and the original reservoir water sample.

9. The method of claim 8, wherein:
a produced water sample comprising only one member selected from the group consisting of the seawater sample, the aquifer water sample, and the original reservoir water sample plots close to its corresponding endpoint;
a produced water sample comprising only two of members selected from the group consisting of the seawater sample, the aquifer water sample, and the original reservoir water sample plots along the outer lines at a position corresponding to an amount of each of the two members; and
a produced water sample comprising the seawater sample, the aquifer water sample, and the original reservoir water sample plots in at a position within the triangle corresponding to an amount of each of the seawater sample, the aquifer water sample, and the original reservoir water sample.

10. The method of claim 1, wherein:
the model comprises endpoints that correspond to mixtures comprising only one member selected from the group consisting of the seawater sample, the aquifer water sample, and the original reservoir water sample;
the model comprises outer lines connecting the endpoints that correspond to mixtures comprising only two members selected from the group consisting of the seawater sample, the aquifer water sample, and the original reservoir water sample;
the outer lines define a triangle; and
the model comprises grid lines that connect the outer lines that correspond to mixtures comprising the seawater sample, the aquifer water sample, and the original reservoir water sample.

11. The method of claim 10, wherein:
a produced water sample comprising only one member selected from the group consisting of the seawater sample, the aquifer water sample, and the original reservoir water sample plots close to the corresponding endpoint;
a produced water sample comprising two members selected from the group consisting of the seawater sample, the aquifer water sample, and the original reservoir water sample plots along the outer lines at a position corresponding to an amount of each of the two members; and
a produced water sample comprising the seawater sample, the aquifer water sample, and the original reservoir water sample plots at a position within the triangle corresponding to an amount of each of the seawater sample, the aquifer water sample, and the original reservoir water sample.

12. The method of claim 1, wherein the seawater has a concentration of chloride of 5000 mg/L to 40000 mg/L.

13. The method of claim 1, wherein the seawater has a δ¹⁸O of −5 to 7.

14. The method of claim 1, wherein the aquifer water has a concentration of chloride of 100 mg/L to 6000 mg/L.

15. The method of claim 1, wherein the aquifer water has a δ¹⁸O of −10 to 3.

16. The method of claim 1, wherein the original reservoir water has a concentration of chloride of 40000 mg/L to 250000 mg/L

17. The method of claim 1, wherein the original reservoir water has a δ¹⁸O of −2 to 8.

18. The method of claim 1, wherein the produced water is from a production well in fluid communication with an underground reservoir.

19. The method of claim 18, wherein the produced water is from a flowback operation.

20. The method of claim 18, wherein the seawater is injected into the underground reservoir.
